(19) United States
(12) Patent Application Publication
(10) Pub. No.: US 2003/0092921 A1
(43) Pub. Date: May 15, 2003

(54) Method of making gold-titania catalyst for oxidation of hydrocarbons

(76) Inventors: Eric E. Stangland, Midland, MI (US); William Nicholas Delgass, West Lafayette, IN (US); Ronald P. Andres, Lafayette, IN (US)

Correspondence Address: Mr. Edward J. Timmer, Walnut Woods Centre, 5955 W. Main Street, Kalamazoo, MI, 49009, US

(21) Appl. No.: 10/159,697

(22) Filed: May 30, 2002

Related U.S. Application Data
(60) Provisional application No. 60/295,931, filed on Jun. 5, 2001.

(51) Int. Cl. B01J 23/52; C07D 301/06

(52) U.S. Cl. 549/533; 502/344; 502/242

(57) ABSTRACT

A catalyst comprises a titanium-containing oxide and gold deposited by deposition-precipitation on the oxide in a gold-bearing solution where the solution and the oxide are centrifuged to separate out the catalyst. The catalyst is used in oxidation of an olefin by contacting the olefin with a reaction gas and the catalyst which is heated in the reaction gas to a reaction temperature at a controlled heating rate to avoid agglomeration of gold species.

This application claims the benefits of U.S. provisional application Serial No. 60/295,931 filed Jun. 5, 2001.

### FIELD OF THE INVENTION

The invention relates to a method and catalyst for the direct oxidation of olefins, such as propylene, by oxygen in the presence of a hydrogen to produce olefin oxides, such as propylene oxide.

### BACKGROUND OF THE INVENTION

The chlorohydrin method, direct organic hydroperoxide oxidation methods such as the Halcon method, and the peracetic acid method have been used as methods of industrial manufacture of propylene oxide (an epoxide). However, these methods are disadvantageous from the standpoint that they produce byproducts and constitute two-stage manufacturing processes.

Although the direct oxidation of ethylene to ethylene oxide is practiced commercially using supported silver catalysts, the direct oxidation of olefins containing allylic hydrogens, such as propylene, generally has exhibited significantly lower selectively to the olefin oxide (epoxide) as a result of side reactions. For example, large amounts of acrolein and oxygen-containing C₁₋₃ byproducts are produced.

U.S. Pat. No. 5,965,754 granted to Clark et al. describes a method of using a vapor-phase oxidation reaction catalyst to oxygen-oxidize an olefin in the presence of hydrogen. This patent describes oxidation of propylene to propylene oxide using a catalyst comprising gold on a support comprising titanium dispersed on silica with the titanium being present as a disorganized phase.

U.S. Pat. No. 5,623,090 granted to Haruta et al. describes a method of using a vapor-phase oxidation reaction catalyst which contains gold and titanium oxide (titania) to oxygen-oxidize a saturated or unsaturated hydrocarbon in the presence of hydrogen. This patent also describes oxidation of propylene to propylene oxide using a gold-titanium oxide-silica catalyst. Despite high selectively to propylene oxide, the patent provides relatively low yields (low conversion of propylene), such as less than 2 mole %, as a result of deactivation and oligomerization effects.

International patent publications WO 00/59632 and WO 00/59633 describe methods for hydro-oxidation of olefins to olefin oxides using a gold catalyst on a support that also contains titanium. While each of these methods provides a means for oxidation of olefins such as propylene to their olefin oxides such as propylene oxide, there is still a need for a catalyst, and method to make such a catalyst, that can provide high selectively to propylene oxide with high conversion of propylene while not rapidly deactivating under reaction conditions

### SUMMARY OF THE INVENTION

The present invention provides a method and catalyst for the direct oxidation of olefins, such as propylene, by oxygen in the presence of molecular hydrogen to produce olefin oxides, such as propylene oxide, with high selectively to olefin oxide and higher conversion of olefins than achieved heretofore. The invention also has the advantage that it can be used at higher temperatures without loss of selectivity due to deactivation. It can also be practiced to epoxidize ethylene and diolefins, such as butadiene, that do not have allylic hydrogens.

In an illustrative embodiment of the present invention, an olefin is contacted with oxygen in the presence of molecular hydrogen and a novel catalyst to produce an olefin oxide. The catalyst comprises a titanium-containing oxide and gold species deposited on the oxide in a manner that improves catalyst performance in terms of providing high selectively to olefin oxide and higher conversion of olefin than achievable heretofore. The catalyst is heated in the reaction gas in the reactor at a controlled, relatively slow rate to improve catalytic activity during the epoxidation reaction. For example, in the conversion of propylene to propylene oxide (PO) yields of 0.2-0.4 grams PO/grams catalyst hour have been achieved with selectivity above 90% at 473 K.

Advantages and objects of the invention will become more readily apparent from the following description.

### DESCRIPTION OF THE DRAWINGS

FIG. 1 is an X-ray photoelectron spectra of the Ti 2p region for anatase, titania-coated silica (TiO₂—SiO₂) and the titanium silicalite TS1(126).

FIGS. 2A and 2B are transmission electron micrographs (TEM's) of catalyst Au(DP)/cTS1(126) before (FIG. 2A) and after (FIG. 2B) the PO reaction.

FIG. 3 is a schematic diagram of the reaction apparatus.

FIG. 4 is an Arrhenius plot of PO TOF versus a temperature parameter for catalysts Au(DP)/cTS1(126) (upper plot) and Au(DP)/uTS1(126) (lower plot).

FIG. 5 is a bar graph showing selectively at 473 K for calcined and uncalcined Au(DP) TS1(126) catalysts.

### DESCRIPTION OF THE INVENTION

Although the invention is described herebelow with respect to direct vapor-phase propylene epoxidation by contacting propylene with oxygen in the presence of molecular hydrogen and in the presence of an improved epoxidation catalyst, the invention is not so limited and can be practiced to effect direct oxidation of olefins having three or more carbon atoms by oxygen in the presence of hydrogen and the improved catalyst to produce olefin oxides with high selectively to the respective olefin oxide and higher conversion of olefins than achieved heretofore. The olefin preferably comprises a C₃₋₁₂ olefin that is converted to the corresponding C₃₋₁₂ olefin oxide. The invention is especially useful in the direct oxidation of propylene to propylene oxide with high selectively to propylene oxide and high conversion of propylene. The invention can be practiced to epoxidize diolefins, such as butadiene, that do not have allylic hydrogens.

The novel epoxidation catalyst used in practice of the invention comprises a titanium-containing oxide and gold deposited by deposition-precipitation (DP) on the oxide in a gold-bearing solution where the solution and the oxide are centrifuged to separate out the DP catalyst followed by thermal activation of the DP catalyst to provide immobilized metallic gold particles or particle clusters on the oxide. Preferably, the titanium-containing oxide is microporous and immobilized gold particles or gold particle clusters reside on exterior surfaces of the oxide and most importantly in micropores of the oxide such that the gold particles residing in the oxide micropores have a diameter less than the micropore diameter, such as about 0.5 nanometers (nm) or less particle diameter.

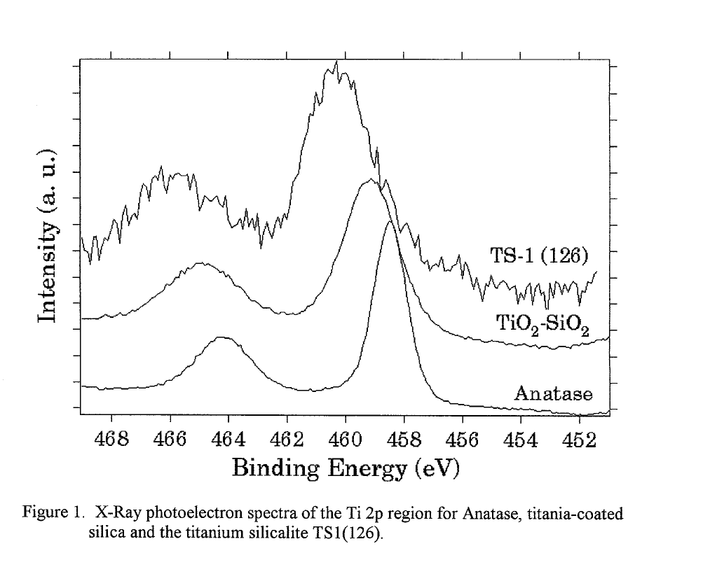

The titanium-containing oxide preferably comprises titanium silicalite where the titanium is primarily in tetrahedral coordination in the oxide lattice and has a positive valence, such as +3 to +4. The titanium is present isolated in the oxide lattice as evidenced by essentially no crystalline titanium dioxide being detected by Raman spectroscopy of the oxide. That is, the peaks associated with the various phases (e.g. rutile, anatase, brookite) of titanium dioxide are not detected by conventional Raman spectroscopy. Furthermore, x-ray diffraction shows the expected orthorhombic structure with Ti incorporated into the silicalite lattice. X-Ray photoelectron spectoscopy, however, shows that the Ti 2p3/2 line for our titanium silicalite (TS1(126)) has a width of 2.6 eV, substantiallly broader than the values of 1.5 eV and 2.1 eV for anatase and titania coated silica (Degussa P25), respectively FIG. 1. The Si 2p line of the silicalite is not affected by charge compensation with the e-gun, so we take the Ti linewidth to indicate a distribution of Ti chemical states. Since the Ti 2p_{3/2} peak position, 460.3 eV, is in good agreement with literature values for TS1 titanium silicalite (B. Notari, Microporous Cystalline Titanium Silicates, Adv. Catal., 41, 253 (1996)), we conclude that the primary state of the Ti is tetrahedral Ti⁴⁺, but that an additional, non-tetrahedral Ti species, perhaps not in the zeolite framework, is also present.

The preferred oxide comprises titanium silicalite having a Si:Ti atom ratio is in the range of 50:1 to 150:1, although other titanium-containing oxides can be used in practice of the invention including, but not limited to monolayer and submonolayer coatings of titanium oxide on silica. Suitable titanium silicalites for use in practice of the invention can be made pursuant to an embodiment of the invention described herebelow in Example 1 where titanium silicalite crystallites are precipitated from a hydroxide reaction solution with crystallite sizes too fine to filter and requiring centrifuging to separate out from the reaction solution followed by calcining of the titanium silicalite crystallite (powder) product. A preferred crystallite or grain size of the calcined titanium silicalite is in the range of 0.01 to 0.1 microns.

As mentioned above, gold is deposited by deposition-precipitation (DP) on the titanium-containing oxide. By deposition-precipitation (DP) is meant a method for causing gold to be deposited and precipitated in the form of a hydroxide or reacted with hydroxyl groups, on the surface of the oxide by controlling the pH and temperature of an aqueous solution of water-soluble gold compound. A DP method is described by Tsubota et al. in “Preparation Of Catalysts V”, Elseiver Science Publishers B. V., Amsterdam, page 695, 1991 and in Haruta et al. U.S. Pat. No. 5,623,090 and Clark et al. U.S. Pat. No. 5,965,754, the teachings of which are incorporated herein by reference.

For purposes of illustrating the invention and not limiting it, deposition-precipitation (DP) of gold on the titanium-containing oxide can be practiced by Na₂CO₃ neutralization to pH 7-9 of a room temperature aqueous HAuCl₄ solution containing or contacting the titanium-containing oxide followed by centrifuging of the solution to separate out the fine DP catalyst solids (i.e. titanium-oxide with gold deposited thereon). The separated catalyst solids can be used directly or washed with water one or more times. The solution alternately can be neutralized by hydroxides including NaOH, CsOH, NH₄O₄H and the like. Alkali hydroxides are preferred over ammonium hydroxide since the decomposition of the ammonium ion can increase surface acidity and thus raise P0 decomposition activity.

The aqueous solution of water-soluble gold compound typically includes an amount of gold that is about 4 to 8 weight % of the amount of finished catalyst, although other Au concentrations can be employed. The DP catalyst solids typically have a weight loading of gold to achieve the desired catalytic effect in the direct oxidation of olefins with oxygen in the presence of hydrogen. The separated catalyst solids then are dried in air or vacuum at a suitable temperature (e.g. 295 K). Typically, the gold loading is about 0.1 to 5.0 weight % of the dry catalyst.

In general, pursuant to an embodiment of the invention, in the direct oxidation of olefins, it is best to thermally treat (activate) the dried catalyst in the reaction gas stream of the reactor by heating slowly to the appropriate reaction temperature of, for example, 423 to 473 degrees C. for propylene-to-propylene oxide conversion. A heating rate of 1 degree C./minute or less is preferred in conducting the propylene-to-propylene oxide reaction. Examples 1 and 2 set forth herebelow describe such thermal treatment to slowly heat the catalyst in the reaction stream.

However, the dried catalyst can be subjected to a precalcination treatment prior to use in the direct oxidation of olefins. The precalcination treatment can be conducted in air or a reducing atmosphere, such as hydrogen, or an inert atmosphere, such as nitrogen and/or He, at a temperature in the range of 573 to 673 K and calcining times in the range of 2 to 6 hours. Calcining can lead to active catalysts, but generally promotes gold particle growth to the detriment of small gold clusters.

The above described catalyst is especially useful in direct vapor-phase olefin epoxidation where the olefin is contacted with oxygen in the presence of molecular hydrogen and the above described catalyst under process conditions effective to produce olefin oxide with high selectively to the olefin oxide and higher conversion of olefins than achieved heretofore.

Generally, the vapor-phase olefin epoxidation can be carried out in a reactor of any conventional design suitable for vapor phase epoxidation such as a fixed bed reactor described herebelow in Example 1 for purposes of illustration and not limitation. The reactants (olefin, hydrogen, oxygen) are contacted with the catalyst at a reaction temperature in the range from about 373 to about 473 K with the optimum temperature for a particular olefin, reactant ratios, and other process variables being readily determined by routine experimentation. The molar ratio of olefin, oxygen, molecular hydrogen and diluent will typically fall within 10:10:10:70 to 10:10:5:75, although other ratios of the reactants may be used. The pressure is typically atmospheric pressure, although the pressure can be varied as desired with a higher pressure expected to increase reaction rate. The space velocity of the feed gas (olefin, hydrogen, oxygen, carrier and optional diluents) through the reactor will generally be in the range of 5 to 20 liter/gram catalyst hour.

The following Examples are offered to further illustrate the invention without in any way limiting it. In the Examples, the term “selectively” is defined as the mole percentage of reacted olefin which forms a particular product, which will include the desired propylene oxide. The term “conversion” is defined as the mole percentage of olefin which reacts to form products. Both a high selectively and high conversion are desired.

### EXAMPLE 1

A) Preparation of Catalyst Ti-Containing Oxide Support

A Ti-containing oxide support comprising titanium silicalite (known as TS-1) was prepared as follows:

A 40 weight % solution of tetraproylammonium hydroxide (12.7 ml) was added dropwise to tetraethylorthosilicate (24.6 ml) in a polypropylene Erlenmeyer flask during vigorous stirring. A solution of titanium (IV) butoxide (1.1 ml) dissolved in iso-propanol (6.2 ml) was then added dropwise to the solution of tetraproylammonium hydroxide and tetraethylorthosilicate. Stirring was continued for another 15 minutes. A final aliquot of tetraproylammonium hydroxide (4.6 ml) was then added dropwise, during which time the mixture began to gel. An additional 20 ml of isopropanol was added to facilitate continued stirring. The yellow solution was then heated in a water bath to 80 degrees C. for 3 hours to remove isopropanol from the solution. Upon such heating, the mixture became caramel colored, but more transparent. After adding 35 ml of distilled water, the solution was then transferred to a sealed Teflon lined pressure vessel and subsequently heated in an oven to 170 degrees C. for 24 hours. The vessel was then removed and allowed to stand at room temperature for one day. The crystallites of TS-1 that formed were too small to be successfully filtered in the laboratory using filter paper capable of capturing particles having a size greater than 6 microns diameter. As a result, the samples of TS-1 were washed and recovered three times using a Fisher Centrific Model 228 benchtop centrifuge operating at 3300 rpm. The procedure was to add 1-2 mls of the TS-1/distilled water preparation mixture to each 8 ml test tube and fill the remainder of each tube with distilled water followed by centrifuging for 30 minutes. The mother liquor was decanted from each test tube, and the test tubes refilled with distilled water and centrifuged again for 30 minutes. The latter procedure was repeated again to provide a total of three centrifugations.

The solid titanium silicalite powder (designated uTS1(126)) thus recovered from the test tubes was dried under vacuum (e.g. 10 milliTorr) at 393K overnight and then calcined in a flow of 20% by volume O₂ in He according to the temperature program: heat from 25 to 120 degrees C. in 1 hour, hold at 120 degrees C. for 1 hour, heat from 120 to 500 degrees C. in 3 hours, hold at 500 degrees C. for 5 hours. The calcined titanium silicalite is designated cTS1(126) hereafter.

A portion of the dried titanium silicalite sample (designated uTS1(126)) was retained and not calcined. The number 126 in parenthesis is the atomic Si:Ti ratio (i.e. Si:Ti=126) in the titanium silicalite zeolite crystallites as determined by atomic absorption testing (Perkin Elmer 3110 atomic absorption spectrometer) of the sample. Characterization of the oxide supports thus made is set forth below in Table I.

TABLE I
Characterization results for catalyst support materials.

| | | TiO₂ | Surface | Micropore | Pore |
|---|---|---|---|---|---|
| | | Loading | Area^{a} | Volume | Diamter^{b} |
| Support | Abbreviation | (wt. %) | (m²/g) | (cm³/g) | (Å) |
| TS-1 | cTSI(126) | 0.6 | 430-610 | 0.12 | 5.2 |
| TS-1 | uTSI(126) | 0.6 | 29-40 | 0.005 | 8.3 |

^{a}Range between BET and Langmuir surface areas.
^{b}Determined by Horvath and Kawzoe method. Diameter at the point of maximum N₂ uptake.

X-ray diffraction (XRD) analysis (using Siemens D500 diffractometer) of the above oxide supports indicated a change from the mono-clinic crystal structure of the silicalite to an orthorombic crystal structure when Ti is incorporated into the silicalite lattice. Raman spectroscopy at a wave length of 785 nm and power of 300 mW performed on the calcined cTS1(126) oxide support showed only a broad line at 400 cm⁻¹ indicative of the silicalite structure. The peak at 960 cm⁻¹ indicative of Ti lattice substitution was not observed, perhaps due to the acquisition time for the sample being insufficient in duration to observe this relatively weak feature at this low Ti loading.

B) Deposition-Precipitation

DP catalysts were prepared by neutralization to pH 7-9 of a room temperature aqueous HAuCl₄ solution having 0.15 weight % Au. In most cases, Na₂CO₃ was used as the neutralizing agent. The DP procedure using Na₂CO₃ as the neutralizing agent was as follows:

One gram samples of uTS1(126) support and cTS1(126) support were each crushed to a fine powder A solution of 0.31 g of HAuCl₄ in 100 ml of distilled water was neutralized to a pH of 7-9 while stirring. 50 ml of solution was poured over each sample and the yellow suspension stirred for 3 hours. Since the particles in the suspensions were too small to filter using filter paper capable of capturing >6 μm particles, the suspensions were recovered and washed by centrifugation. The centrifuge tubes were then filled with the suspensions and centrifuged for 30 minutes. using a Fisher Centrific Model 228 benchtop centrifuge operating at 3300 rpm. After the mother liquor was decanted, 5-7 ml of distilled water was added to each tube, and the suspensions were centrifuged again for 30 minutes. This washing step was repeated two more times to provide a total of three washing steps. The recovered DP catalyst powders were dried under vacuum (e.g. 10 milliTorr) at 373 K overnight or 295 K for one day with the room temperature drying being preferred. These catalysts are designated Au(DP)/uTS1(126) or Au(DP)/cTS1(126).

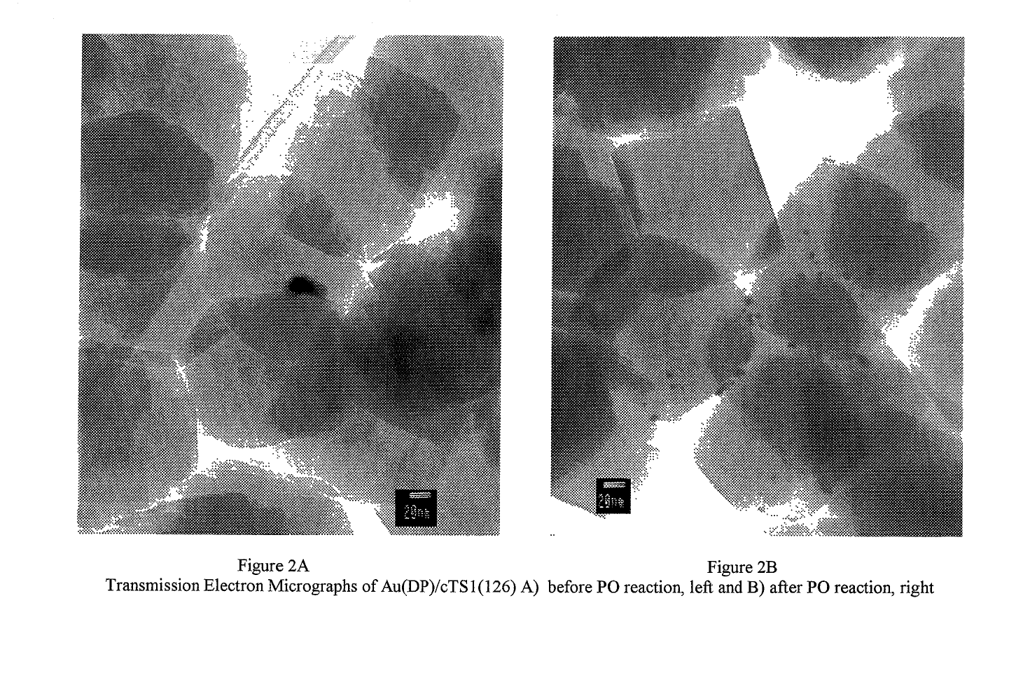

FIGS. 2A and 2B are transmission electron micrographs (TEM's using JEOL 2000 FX electron microscope) of the DP catalyst cTS1(126) before and after use in the propylene-to-propylene oxide reaction as described below. Essentially no particles are seen in the as-prepared catalyst (before reaction). The small particles in the 1-5 nm range seen after the reaction are indicative of particle growth during reaction. Those particles reside outside the zeolite channels.

BET analysis confirmed that the pore volume of Au(DP)/uTS1(126) catalyst increased 10 times after the propylene-to-propylene oxide reaction from 0.0006 cm³/gram to 0.006 cm³/gram, indicating that the template (tetraproylammonium hydroxide having tetraproylammonium cation around which the TS-1 structure was formed) was partially removed during the reaction. BET analysis (named for Brunauer, Emmett, and Teller) is a known analysis of surface area and pore volume from physical adsorption of N₂ at 77K.

The pore volume of the uncalcined Au(DP)/uTS1(126) catalyst was still 10 times lower than that of the calcined Au(DP)/cTS1(126) catalyst after the PO reaction. The pore volume of the Au(DP)/cTS1(126) catalyst decreased after propylene-to-propylene oxide reaction from 0.12 cc³/gram to 0.07 cc³/gram of catalyst presumably due to pore blockage with carbonaceous species.

The amount of gold deposited on the oxide support is significantly less than that in the original HAuCl₄ solution as shown in Table II below. For example, see the gold loading values in Table II.

TABLE II
Characterization results for various deposition-precipitation catalysts.

| | | Gold Loading | Dₚ |
|---|---|---|---|
| Catalyst | Preparation Method | (wt. %) | (nm) |
| Au(DP)/cTS1(126) | Au DP with Na₂CO₃ | 0.3 | 5.9 ± 3.4 |
| | on calcined TS1(126) | | |
| Au(DP)/uTS1(126) | Au DP with Na₂CO₃ | 0.2 | 10.8 ± 4.4 |
| | on uncalcined | | |
| | TS1(126) | | |

The average gold particle size measured after PO reaction, Dₚ (nm) in Table II, was determined by measuring at least 50 particles from TEM micrographs using Optimum Version 6.1 image analysis software. It is not surprising that the gold particle size is larger for the uncalcined support since the internal volume of the crystallites is blocked and the gold can only attach to the external surface. It is also noted that gold clusters too small to be seen by these TEM methods may be at least as important as the large gold particles as active sites in this catalyst.

C) Propylene to Propylene Oxide Kinetic Tests

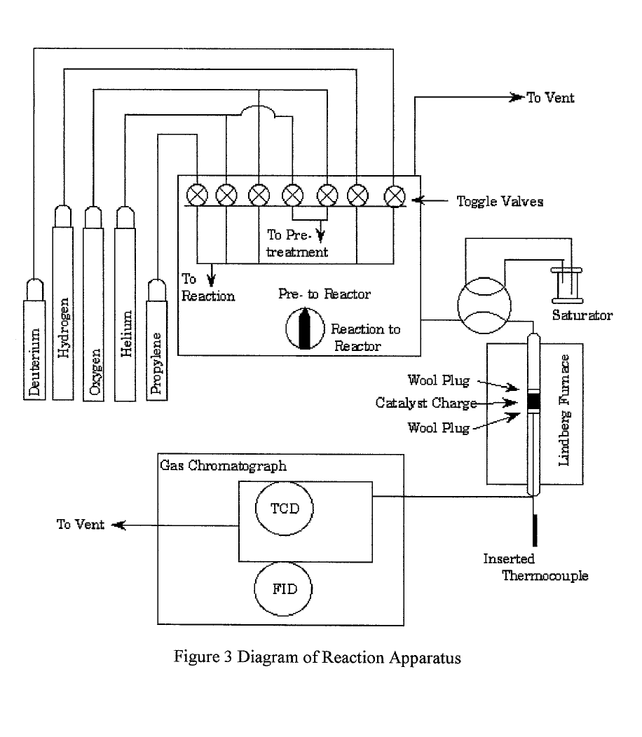

A ½ inch diameter stainless steel reactor was used with the catalyst charge (fixed bed) packed therein between two glass wool plugs, FIG. 3. The charge weight for the Au(DP)/cTS1(126) catalyst (calcined) was 0.31 grams. The charge weight for the Au(DP)/uTS1(126) catalyst (uncalcined) was 0.22 grams, see Table II.

A K-type thermocouple used to control reactor temperature was passed through the catalyst bed from the bottom and rested at the top edge of the catalyst charge.

The catalysts were slowly heated up to reaction temperature in the reaction stream in the reactor in order to produce the highest catalytic activities pursuant to an embodiment of the invention. For example, the heating rate of the catalyst in the reactor is controlled slow enough to avoid agglomeration of the gold clusters while still thermally activating the catalyst. In this Example, the catalyst was slowly heated at a heating rate of 1 degree C./minute in the reaction stream comprising 10/10/10/70 volume % of propylene (99.9% pure) O₂, H₂ (99.9995% pure) and He carrier, respectively.

Reactant concentrations used for the standard propylene oxidation experiment were 10/10/10/70 volume % of propylene (99.9% pure) O₂, H₂ (99.9995% pure) and He carrier, respectively. All gases were used without further purification. A diagram of the reactor apparatus is illustrated schematically in FIG. 3.

Catalyst activity was tested for the sample Au(DP)/cTS1(126) by a temperature program profile consisting of 473-413433-373K, holding for 6 hours at each temperature and taking samples at 35 minute intervals. After this program, the 473 K temperature was immediately re-tested for approximately 3 hours. Catalyst activity was tested for the sample Au(DP)/uTS1(126) by a temperature program profile consisting of 373443-413-473K, holding for 6 hours at each temperature and taking samples at 35 minute intervals. After this program, the 443 K temperature was immediately re-tested for approximately 3 hours. As mentioned, the heating rate to 373K was 1 degrees C./minute.

As shown in FIG. 3, effluent samples from the reactor were analyzed by a Varian 3740 gas chromatograph using He carrier gas, a Chromosrob 102 packed column (Supelco, Inc.) and a thermal conductivity detector (TCD) to monitor H₂, O₂, CH₄, propylene, and propane, and a Supelcowax 10 capillary column (Supelco, Inc.) with an flame ionization detector (FID) to monitor ethanol, propylene oxide, propanol, acetone, acrolein, and traces of other unidentified oxygenates. The He carrier gas maximizes TCD sensitivity and detection of trace components, but promotes non-linearity of the H₂ response factor due to H₂/He mixture thermoconductivity.

Turnover frequencies (TOF's) were calculated based on the determination of an apparent gold particle surface area using the Dₚ obtained from TEM's and the atomic absorption results for the Au weight loading. A hemispherical particle model and a Au metal density of 19.3 grams/cc were assumed giving equation 3.1:

$$SA = \frac{( 6 ) \text{(} wt . \% \text{/} 100 \text{)}}{\text{(} {10}^{6} \text{<} {D}_{p} \text{>} {ρ}_{Au} \text{)}} L \qquad [ 3.1 ]$$

where:

SA=Total gold surface area (Au atoms g⁻¹)

wt. %=Catalyst loading in weight percent

<Dₚ>=Average particle diameter (nm) as measured by TEM

ρ=Bulk density of Au=19.3 μm³

L=1.146×10¹⁹ Au atoms per m², as determined by an average over the low index faces

TOF's were used to calculate selectivity to product “i” defined as vᵢ(TOF)/ vₖ(TOFₖ) where the denominator is the sum of TOF's for the “k” oxidation products observed and “v” is the appropriate inverse of the stoichiometric coefficient. The representative equation for calculating TOF data from the gas chromatograph (GC) data is shown in equation 3.2:

$${r}_{1} = ( \begin{matrix}{A}_{i} & {v}_{0} & N \\ 100 \% & {R}_{i} & W\end{matrix} ) ( SA ) \qquad [ 3.2 ]$$

where:

rᵢ=TOF (molecules species i (gold atoms)⁻¹s⁻¹)

Aᵢ=Integrated Peak area from GC

v₀=Volumetric flow rate (STP ml min⁻¹)

Rᵢ=GC response factor (area per %)

W=Catalyst charge weight (g)

N=4.479×10¹⁹ (conversion factor)

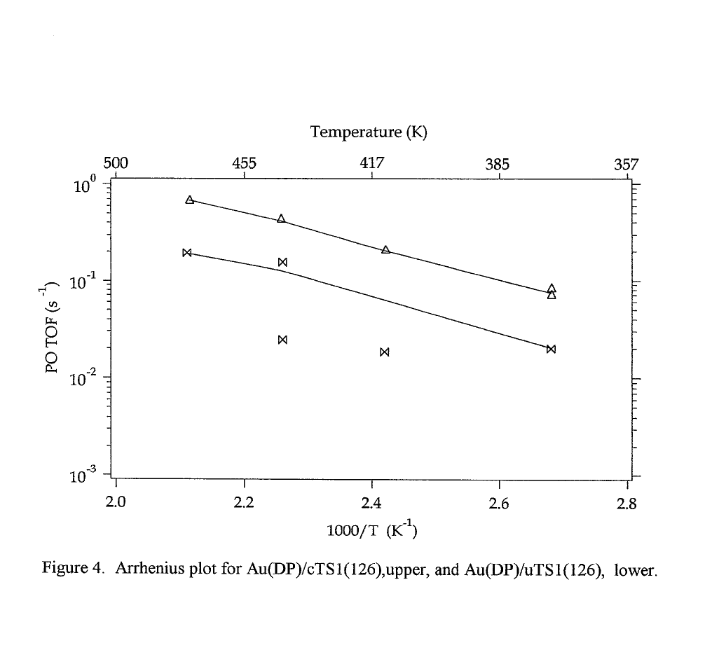

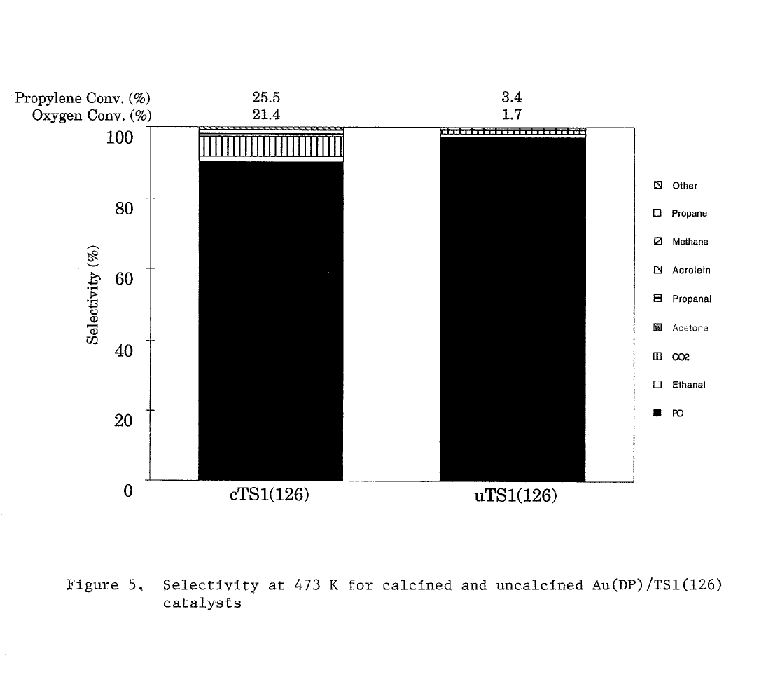

The results of the kinetic testing are set forth in FIGS. 4 and 5. FIG. 4 is a set of Arrhenius type plots of PO activity, while FIG. 5 comprises a set of stacked bar graph plots showing product selectivities with PO being the dark band at the bottom of each stack. The stack plots show product selectivities at 473K where selectively differences are more pronounced. Propylene conversion (mole %) and oxygen conversion (mole %) are set forth at the top of the stacked bar graphs.

FIGS. 4 and 5 reveal that the Au(DP)/cTS1(126) catalyst and Au(DP)/uTS1(126) catalyst provided greater than 90% PO selectivity at 473K. Similar high selectively (greater than 90%) was observed at the other temperatures tested.

Importantly, the propylene conversion of the Au(DP)/cTS1(126) catalyst at 473K was greater than 20 mole %; namely, 25.5 mole %. In alternative units, this result corresponds to a propylene yield of 0.4 grams PO/gram catalyst-hour. In contrast, the propylene conversion of the Au(DP)/uTS1(126) catalyst at 473K was only 1.7 mole %, comparable to conversion percentages achieved heretofore.

The achievement of both high selectivity to propylene oxide (PO) and high conversion of propylene of 20 mole % and greater achieved by the Au(DP)/cTS1(126) catalyst is an unexpected and dramatic improvement over previously reported catalysts, especially the 2-3 mole % conversion or yield cap due proposed by Nijhuis et al. in Direct Epoxidation of Propene using Gold Dispersed on TS-1 and other Titanium-Containing Supports, Ind. Eng. Chem. Res. 38, 884-891 (1999) or the 3-6% conversion reported by B. Uphade, M. Okumura, N. Yamada, S. Tsubota and M. Haruta, Stud. Surf. Sci. Catal., 130A, 833 (2000).

Another important characteristic of the Au(DP)/cTS1(126) catalyst is the fact that the PO rate is still rising as temperature is increased from 373 to 473K. Most conventional catalysts have a rate maximum with temperature within that range. This behavior (rising PO rate as temperature is increased from 373 to 473K) allows high selectively at high enough temperatures to avoid catalyst fouling by adsorbed or polymerized particles.

Although the uncalcined Au(DP)/uTS1(126) catalyst was prepared from the same reaction solution as the calcined Au(DP)/cTS1(126) catalyst, the uncalcined catalyst exhibited a PO TOF one order of magnitude (i.e. 10 times) lower than that of the calcined catalyst. However, the selectivity of the calcined and uncalcined catalysts was similar. It is interesting to note that the difference in PO TOF for the uncalcined Au(DP)/uTS1(126) catalyst and the calcined Au(DP)/cTS1(126) catalyst after a full cycle of exposure at 473K was roughly a factor of 10, the same factor describing the difference in pore volume between the two catalysts. This is a strong indication that gold particles less than 6 Angstroms inside the pores of the cTS1(126) oxide support are the active gold species, although the inventors do not wish to be bound by any theory in this regard.

### EXAMPLE 2

A) Preparation of Catalyst Ti-Containing Oxide Support

The unused portion of the uncalcined TS-1(uTS1(126)) from Example 1 was used as the support in this example. Approximately 0.4210 grams of uTS1(126) was calcined in accordance with the procedure set forth in example 1. No characterization of the calcined support was conducted.

B) Deposition-Precipitation

DP catalysts were prepared by neutralization to pH 7-9 of a room temperature aqueous HAuCl₄ solution having 0.0955 grams HAuCl₄ in 30 mls of distilled water. Na₂CO₃ was used as the neutralizing agent as described in Example 1. The solution was then added to 0.4 grams of the TS1 support. The suspension was then stirred for 4 hours and then separated equally into four test tubes and centrifuged in the centrifuge described above for 30 minutes. Three washes of the TS1 support were accomplished by adding about 5 ml of distilled water to each vial followed by 30 minutes of centrifugation. The catalyst was then dried overnight at room temperature in a vacuum oven. The resulting dried catalyst was used for kinetic measurements.

C) Propylene to Propylene Oxide Kinetic Tests

Using the reaction set-up described for Example 1, 0.163 g of the dried catalyst was placed in the reactor between two glass wool plugs, FIG. 3. The reactor was then purged for 30 minutes at room temperature in a 20/80 mixture of O₂/He (by volume) at approximately 50 ml/minute. A reaction mixture containing H₂/O₂/C₃H/1e at a ratio of 10/10/10/70 at a flow rate of 35 ml/minute was introduced at room temperature. The catalyst was then slowly heated in the reaction mixture (reaction gas) from room temperature to 373K over a period of 1 1/2 hours. A temperature program of 373-413-443-473-373-413-443-473K was used for kinetic analysis. At each step, the temperature was held constant for a period of 6 hours while gas chromatographic measurements were made every 35 minutes. In the first heating cycle from 373 to 473 K, the catalyst did not reach steady state. In the second cycle, the conversion was only 0.4% at 373K, but it rose to 2.04% at 413K and 3.53% at 443K. At 473 K, the conversion of propylene to PO was 6.12%, corresponding to a yield of 0.20 g PO/g catalyst hour. The catalyst was then removed and mixed with 0.2068 grams of fresh catalyst from the same batch to give a total of 0.3668 grams of catalyst. That catalyst charge produced conversions of 0.94%, 3.57%, 6.26%, and 9.83% in the first cycle of 373, 413, 443, and 473K. The 9.835 conversion to PO at 473K corresponds to a yield of 0.15 g PO/g catalyst hour. In the second temperature cycle, the conversion tracked well at 413 and 443K, but at 473k dropped to 2.39%.

Although the invention has been described with respect to certain specific embodiments for purposes of illustration, those skilled in the art will appreciate that changes, modifications, and the like can be made to the embodiments within the scope of the invention as set forth in the appended claims.

1. A catalyst, comprising a titanium-containing oxide and gold deposited by deposition-precipitation on the oxide in a gold-bearing solution where the solution and the oxide are centrifuged to separate out the catalyst.

2. The catalyst of claim 1 wherein the oxide is porous.

3. The catalyst of claim 2 wherein gold particles reside in pores of the oxide such that the gold particles have a diameter less than the diameter of the pores.

4. The catalyst of claim 3 wherein gold particles also reside on the exterior surfaces of the oxide.

5. The catalyst of claim 1 wherein the titanium-containing oxide comprises titanium silicalite.

6. The catalyst of claim 5 wherein the titanium is present isolated in the oxide lattice as evidenced by the lack of crystalline titanium dioxide being detected by Raman spectroscopy, by x-ray diffraction showing evidence of tetrahedral Ti in the lattice, and by evidence from x-ray photoelectron spectroscopy for tetrahedral and non-tetrahedral Ti species.

7. The catalyst of claim 5 wherein the titanium silicalite has a Si:Ti atom ratio is in the range of 50:1 to 150:1.

8. In a method where an olefin is contacted by oxygen in the presence of hydrogen to produce an olefin oxide, the improvement for increasing conversion of the olefin comprising conducting the contacting step in the presence of the catalyst of claim 1.

9. In a method where an olefin is contacted by oxygen in the presence of hydrogen to produce an olefin oxide, the improvement for maintaining conversion of the olefin and high selectivity to PO at temperatures of greater than or equal to 473K comprising conducting the contacting step in the presence of the catalyst of claim 1.

10. A method of making a catalyst for use in oxidation of an olefin in the presence of hydrogen, comprising providing a titanium-containing oxide, depositing gold on the oxide by deposition-precipitation in a gold-bearing solution where the solution and the oxide are centrifuged to separate out the catalyst.

11. The method of claim 10 including heating the catalyst before the oxidation reaction at a heating rate of not greater than 1 degree per minute from 295 K to the reaction temperature while the reaction gas is flowing over the catalyst.

12. The method of claim 9 wherein the oxide is precipitated from a reaction solution followed by centrifuging to separate out oxide crystallites from the reaction solution.

13. The method of claim 12 including heating the catalyst at a rate of not greater than 1 degree C. per minute from 295 K to the reaction temperature while the reaction gas is flowing over the catalyst to increase PO activity.

14. In a method where an olefin is contacted by a reaction gas and a catalyst having gold species thereon in a reactor to produce an olefin oxide, the improvement comprising heating the catalyst in the reaction gas to a reaction temperature at a controlled heating rate to avoid agglomeration of the gold species.

15. The method of claim 14 wherein the catalyst is heated at a rate of 1 degree C./minute or less.
